(19) United States
(12) Patent Application Publication
(10) Pub. No.: US 2025/0062004 A1
(43) Pub. Date: Feb. 20, 2025

(54) METHOD AND APPARATUS FOR DETERMINING CALORIC INTAKE OF USER, ELECTRONIC DEVICE AND STORAGE MEDIUM

(71) Applicant: Beijing Zitiao Network Technology Co., Ltd., Beijing (CN)

(72) Inventors: Jialiang LI, Beijing (CN); Shanping ZHONG, Beijing (CN); Feng ZHU, Beijing (CN)

(21) Appl. No.: 18/720,601

(22) PCT Filed: Dec. 23, 2022
(86) PCT No.: PCT/CN2022/141382

§ 371 Date: Jun. 14, 2024

(30) Foreign Application Priority Data
Jan. 4, 2022 (CN) 202210044916.1

(51) Int. Cl. G16H 20/60

(52) U.S. Cl. CPC G16H 20/60

(57) ABSTRACT

The present disclosure relates to a method for determining caloric intake of a user, and an electronic device and a storage medium. The method includes: acquiring preprandial picture information of food, and determining a preprandial caloric value of the food based on the preprandial picture information of the food; presenting a preprandial picture of the food annotated with the preprandial caloric value of the food; acquiring postprandial picture information of the food, and determining a postprandial caloric value of the food based on the postprandial picture information of the food; determining a caloric intake value of a user based on the preprandial caloric value and the postprandial caloric value of the food; and presenting a postprandial picture of the food annotated with one or more of the preprandial caloric value of the food, the postprandial caloric value of the food, or the caloric intake value of the user.

### CROSS-REFERENCE TO RELATED APPLICATIONS

The present application is based on and claims the priority to the Chinese Application No. 202210044916.1 filed on Jan. 14, 2022, the disclosure of which is incorporated by reference herein in its entirety.

### TECHNICAL FIELD

The present disclosure relates to the technical field of artificial intelligence, and in particular, to a method and apparatus for determining caloric intake of a user, an electronic device and a storage medium.

### BACKGROUND

Health of the human body is closely related to daily eaten food, so that calories of the food are very important for weight management and health. In the related art, when a user records calories he/she obtains through food by using a health management application, he/she needs to look up, by himself/herself, calories of the food he/she eats, and then calculate calories he/she takes in and input the calories to the health management application by himself/herself.

### SUMMARY

The present disclosure provides a method and apparatus for determining caloric intake of a user, and an electronic device and a storage medium.

In a first aspect, the present disclosure provides a method for determining caloric intake of a user, comprising:
acquiring preprandial picture information of food, and determining a preprandial caloric value of the food based on the preprandial picture information of the food;
presenting a preprandial picture of the food, wherein the preprandial picture of the food is annotated with the preprandial caloric value of the food;
acquiring postprandial picture information of the food, and determining a postprandial caloric value of the food based on the postprandial picture information of the food;
determining a caloric intake value of a user based on the preprandial caloric value and the postprandial caloric value of the food; and
presenting a postprandial picture of the food, wherein the postprandial picture of the food is annotated with one or more of the preprandial caloric value of the food, the postprandial caloric value of the food, or the caloric intake value of the user.

In a second aspect, the present disclosure further provides an apparatus for determining caloric intake of a user, comprising:
a first determination module configured to acquire preprandial picture information of food, and determine a preprandial caloric value of the food based on the preprandial picture information of the food;
a first presentation module configured to present a preprandial picture of the food, wherein the preprandial picture of the food is annotated with the preprandial caloric value of the food;
a second determination module configured to acquire postprandial picture information of the food, and determine a postprandial caloric value of the food based on the postprandial picture information of the food;
a third determination module configured to determine a caloric intake value of a user based on the preprandial caloric value and the postprandial caloric value of the food; and
a second presentation module configured to present a postprandial picture of the food, wherein the postprandial picture of the food is annotated with one or more of the preprandial caloric value of the food, the postprandial caloric value of the food, or the caloric intake value of the user.

In a third aspect, the present disclosure further provides an electronic device, comprising:
one or more processors; and
a storage means configured to store one or more programs,
the one or more programs, when executed by the one or more processors, causing the one or more processors to implement the method for determining caloric intake of a user as described above.

In a fourth aspect, the present disclosure further provides a computer-readable storage medium having thereon stored a computer program which, when executed by a processor, implements the method for determining caloric intake of a user as described above.

In a fifth aspect, the present disclosure provides a computer program, comprising: instructions which, when executed by a processor, cause the processor to perform any of the methods for determining caloric intake of a user according to embodiments of the present disclosure.

### BRIEF DESCRIPTION OF THE DRAWINGS

The accompanying drawings herein, which are incorporated in and constitute a part of this specification, illustrate embodiments consistent with the present disclosure and, together with the description, serve to explain the principles of the present disclosure.

In order to more clearly illustrate the technical solutions in the embodiments of the present disclosure, the drawings that need to be used in the description of the embodiments will be briefly described below, and it is obvious that for one of ordinary skill in the art, other drawings can also be obtained without paying creative efforts.

FIG. 1 is a flow diagram of a method for determining caloric intake of a user according to an embodiment of the present disclosure;

FIG. 2 is a presentation effect diagram of a preprandial picture of food according to an embodiment of the present disclosure;

FIG. 3 is a presentation effect diagram of a postprandial picture of food according to an embodiment of the present disclosure;

FIG. 4 is a flow diagram of a method for implementing S130 according to an embodiment of the present disclosure;

FIG. 5 is a schematic diagram of a shooting interface according to an embodiment of the present disclosure;

FIG. 6 is a schematic diagram of another shooting interface according to an embodiment of the present disclosure;

FIG. 7 is a flow diagram of another method for implementing S130 according to an embodiment of the present disclosure;

FIG. 8 is a schematic diagram of another shooting interface according to an embodiment of the present disclosure;

FIG. 9 is a schematic diagram of another shooting interface according to an embodiment of the present disclosure;

FIG. 10 is a schematic diagram of another shooting interface according to an embodiment of the present disclosure;

FIG. 11 is a schematic structural diagram of an apparatus for determining caloric intake of a user according to an embodiment of the present disclosure;

FIG. 12 is a schematic structural diagram of an electronic device according to an embodiment of the present disclosure.

### DETAILED DESCRIPTION

In order that the above objectives, features and advantages of the present disclosure can be more clearly understood, solutions of the present disclosure will be further described below. It should be noted that, without conflict, the embodiments of the present disclosure and features in the embodiments may be combined with each other.

In the following description, numerous specific details are set forth in order to provide a thorough understanding of the present disclosure, but the present disclosure may be implemented in other ways different from those described herein; and it is obvious that the embodiments in the description are only some of the embodiments of the present disclosure, rather than all of them.

In the related art, when a user records calories he gets from food by using a health management application, he/she needs to look up, by himself/herself, calories of the food he eats, and then calculate calories he/she takes in and input the calories into the health management application by himself/herself, so that the whole process is cumbersome and time-consumed, and is not conducive to keeping records by the user.

In order to solve the above technical problem, or at least partially solve it, the present disclosure provides a method and apparatus for determining caloric intake of a user, an electronic device and a storage medium.

Compared with the related art, the technical solution provided in the embodiment of the present disclosure has the following advantages: according to the technical solution provided in the embodiment of the present disclosure, by acquiring preprandial picture information of food, and determining a preprandial caloric value of the food based on the preprandial picture information of the food; acquiring postprandial picture information of the food, and determining a postprandial caloric value of the food based on the postprandial picture information of the food; and determining a caloric intake value of a user based on the preprandial caloric value and the postprandial caloric value of the food, the caloric intake value of the user can be calculated quickly and accurately, and the whole determination process is simple and less time-consumed, which is conducive to helping the user keep records of the caloric intake value, so as to help the user realize weight or health management and improve user experience.

According to the technical solution provided in the embodiment of the present disclosure, by presenting a preprandial picture of the food, wherein the preprandial picture of the food is annotated with the preprandial caloric value of the food; and presenting a postprandial picture of the food, wherein the postprandial picture of the food is annotated with one or more of the preprandial caloric value of the food, the postprandial caloric value of the food or the caloric intake value of the user, it can help the user visually know about the caloric value of the food and/or the caloric intake value of the user.

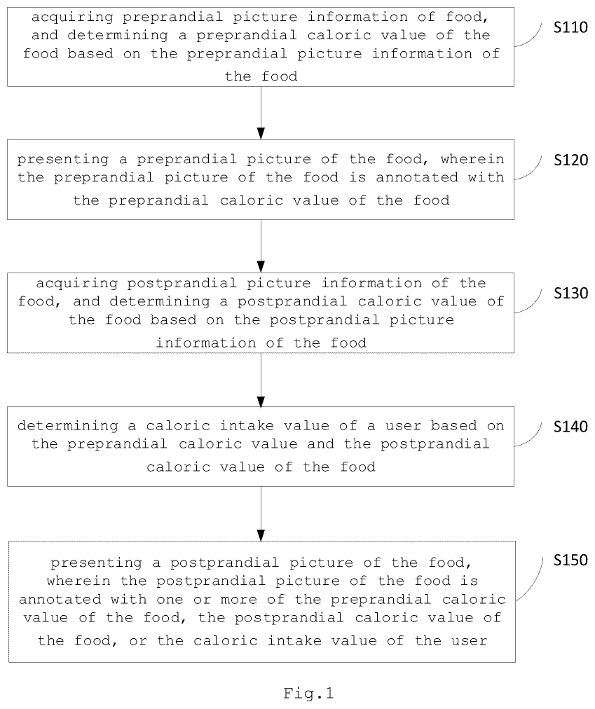

FIG. 1 is a flow diagram of a method for determining caloric intake of a user according to an embodiment of the present disclosure. This embodiment is applicable to a case where the caloric intake of a user is determined or recorded in a client. The method may be performed by an apparatus for determining caloric intake of a user. The apparatus may be implemented in software and/or hardware, and may be configured in an electronic device, for example, a terminal, specifically comprising but not limited to a smartphone, a palmtop, a tablet, a wearable device with a display screen, a desktop computer, a notebook, an all-in-one computer, a smart home appliance, and the like.

Referring to FIG. 1, the method comprises: S110, acquiring preprandial picture information of food, and determining a preprandial caloric value of the food based on the preprandial picture information of the food.

There are various specific implementations for the “acquiring preprandial picture information of food”, which are not limited in the present application. Exemplarily, in response to a first operation for collecting a preprandial picture of food, a shooting interface is presented; and in response to a second operation for collecting the preprandial picture of the food, a display picture of the shooting interface is saved as the preprandial picture information of the food. The first operation is used for triggering the terminal to enter a picture collection mode, and the second operation is used for triggering the terminal to save the picture displayed by a display screen of the terminal, that is, picture collection is completed. In some embodiments, the terminal displays a SHOOT control, and when the user clicks on the SHOOT control, the terminal displays the shooting interface. The user aims a terminal camera at the food to be shot so that the food to be shot appears in the terminal display screen. The user clicks on a SAVE control in the shooting interface so that the terminal saves the picture displayed by the terminal display screen.

There are various specific implementations for the “determining a preprandial caloric value of the food based on the preprandial picture information of the food”, which are not limited in the present application. Exemplarily, preprandial information of the food comprised in the picture is determined based on the preprandial picture information of the food, the preprandial information of the food comprising a name of the food and a preprandial weight of the food; and the preprandial caloric value of the food is determined based on the preprandial information of the food.

Further, the determining preprandial information of the food comprised in the picture based on the preprandial picture information of the food may comprise: pre-constructing a food database, the food database comprising a plurality of pieces of information, each comprising a name of a food, a calorific value per unit weight and density of the food, and a picture of the food; comparing the preprandial picture of the food with the information in the food database to obtain data hit (namely matched) with the food in the preprandial picture of the food; determining the name, caloric value per unit weight and density of the food comprised in the preprandial picture of the food based on the hit data; obtaining size information of the food in the preprandial picture of the food based on the preprandial picture of the food, and obtaining volume information of the food based on the size information of the food; and obtaining a weight of the food based on the volume information and the density of the food, and obtaining the caloric value of the food based on the weight and caloric value per unit weight of the food. In some embodiments, the preprandial size information of the food is obtained from the preprandial picture of the food using a depth of field calculation technique.

Further, “determining information of the food comprised in the picture based on the preprandial picture information of the food, the information of the food comprising a name of the food and a weight of the food” may comprise: acquiring purchase information of the food; determining the information of the food based on the purchase information of the food and the preprandial picture information of the food, the information of the food comprising the name of the food, a brand of the food, and the weight of the food. The purchase information is purchase information generated by the user in at least one of a restaurant, a takeaway platform, a supermarket or a convenience store. The purchase information comprises the brand, name, and weight of the food the user purchases. The reason for this setting is that in practice, calorific values per unit weight may be different for same foods from different brands. When the food information is determined, by combining the purchase information of the food, the caloric value of the determined food is made more accurate, so as to make subsequently obtained caloric intake values of the user more accurate.

The method further comprises: S120, presenting the preprandial picture of the food, wherein the preprandial picture of the food is annotated with the preprandial caloric value of the food.

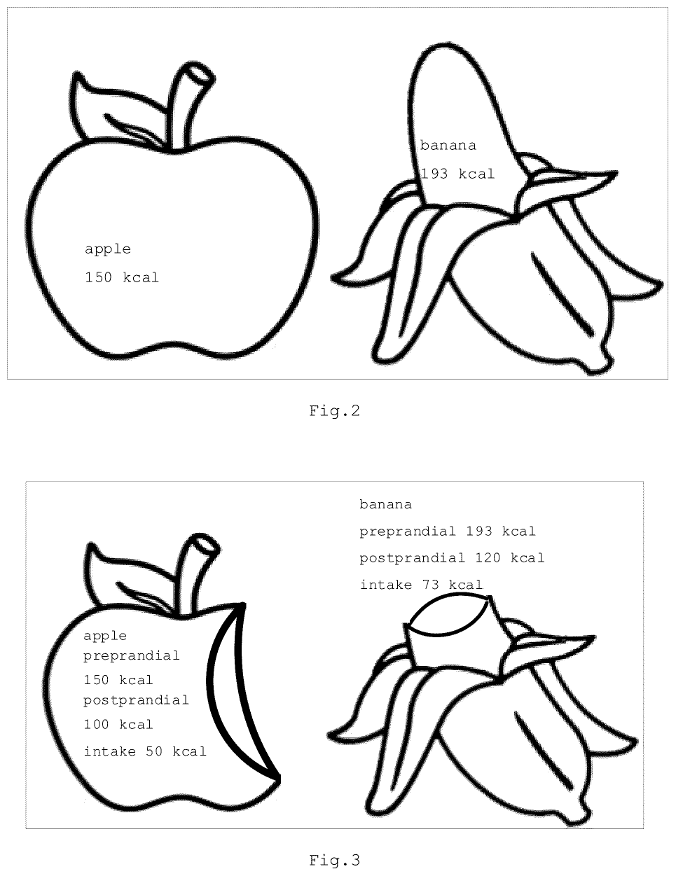

FIG. 2 is a presentation effect diagram of a preprandial picture of food according to an embodiment of the present disclosure. Referring to FIG. 2, the preprandial picture of the food comprises an apple and a banana, the vicinity of the apple being annotated with a preprandial caloric value of the apple, and the vicinity of the banana being annotated with a preprandial caloric value of the banana.

The method further comprises: S130, acquiring postprandial picture information of the food, and determining a postprandial caloric value of the food based on the postprandial picture information of the food.

There are various specific implementations for the “acquiring postprandial picture information of the food”, which are not limited in the present application. Exemplarily, in response to a first operation for collecting a postprandial picture of the food, a shooting interface is presented; and in response to a second operation for collecting a postprandial picture of the food, a display picture of the shooting interface is saved as postprandial picture information of the food. The first operation is used for triggering the terminal to enter a picture collection mode. The second operation is used for triggering the terminal to save the picture displayed by a display screen of the terminal, that is, picture collection is completed. In some embodiments, the terminal displays a SHOOT control, and when the user clicks on the SHOOT control, the terminal displays a shooting interface. The user aims a terminal camera at the food to be shot so that the food to be shot appears in the display screen of the terminal. The user clicks on a SAVE control in the shooting interface so that the terminal saves the picture displayed by the display screen of the terminal.

There are various specific implementations for the “determining a postprandial caloric value of the food based on the postprandial picture information of the food”, which is not limited in the present application. Exemplarily, postprandial information of the food comprised in the picture is determined based on the postprandial picture information of the food, the postprandial information of the food comprising a name of the food and a postprandial weight of the food; and the postprandial caloric value of the food is determined on basis of the postprandial information of the food.

Further, the determining postprandial information of the food comprised in the picture based on the postprandial picture information of the food may comprise: pre-constructed a food database, the food database comprising a plurality of pieces of information, each comprising a name of a food, a calorific value per unit weight and density of the food, and a picture of the food; comparing the postprandial picture of the food with the information in the food database to obtain data hit with the food in the postprandial picture of the food; determining the name, caloric value per unit weight and density of the food comprised in the postprandial picture of the food based on the hit data; obtaining size information of the food in the postprandial picture of the food based on the postprandial picture of the food, and obtaining volume information of the food based on the size information of the food; and obtaining a weight of the food based on the volume information and the density of the food, and obtaining the caloric value of the food based on the weight and the caloric value per unit weight of the food.

The method further comprises: S140, determining a caloric intake value of the user based on the preprandial caloric value and the postprandial caloric value of the food.

There are various specific implementations of this step, which are not limited in the present application. In some embodiments, a difference between the preprandial caloric value and the postprandial caloric value of the food is taken as the caloric intake value of the user.

Further, the caloric intake value of the user can be corrected with an error parameter, and the corrected caloric intake value of the user is taken as a final caloric intake value of the user. The error parameter may be determined based on a terminal camera parameter, or another parameter, which is not limited in the present application.

The method further comprises: S150, presenting a postprandial picture of the food, wherein the postprandial picture of the food is annotated with one or more of the preprandial caloric value of the food, the postprandial caloric value of the food or the caloric intake value of the user.

FIG. 3 is a presentation effect diagram of a postprandial picture of food according to an embodiment of the present disclosure. Referring to FIG. 3, the postprandial picture of the food comprises an apple and a banana, the vicinity of the apple being annotated with a preprandial caloric value of the apple, a postprandial caloric value of the apple and a caloric intake value of the user, and the vicinity of the banana being annotated with a preprandial caloric value of the banana, a postprandial caloric value of the banana and a caloric intake value of the user.

According to the above technical solution, by acquiring preprandial picture information of food, and determining a preprandial caloric value of the food based on the preprandial picture information of the food; acquiring postprandial picture information of the food, and determining a postprandial caloric value of the food based on the postprandial picture information of the food; and determining a caloric intake value of a user based on the preprandial caloric value and the postprandial caloric value of the food, the caloric intake value of a user can be quickly and accurately calculated, and the whole determination process is simple and less time-consumed, which is conducive to helping the user keep records of the caloric intake value, so as to help the user realize weight or health management and improve user experience.

Moreover, according to the technical solution, by presenting a preprandial picture of the food, wherein the preprandial picture of the food is annotated with the preprandial caloric value of the food; and presenting a postprandial picture of the food, wherein the postprandial picture of the food is annotated with one or more of the preprandial caloric value of the food, the postprandial caloric value of the food or the caloric intake value of the user, it can help the user visually know about the caloric value of the food and/or the caloric intake value of the user.

In specific examples of the above technical solution, the method of “determining postprandial information of the food comprised in the picture based on the postprandial picture information of the food” is the same as the method of “determining preprandial information of the food comprised in the picture based on the preprandial picture information of the food”, both of which are implemented by means of the food database. If the preprandial picture of the food comprises N foods (N is an integer greater than or equal to 2), since preprandial and postprandial forms of the food would change, with the above examples, for both each food in the preprandial picture of the food and each food in the postprandial picture of the food, the food database must be queried, that is, in the whole process of determining the caloric intake value of the user, the food database must be queried 2N times in total.

To further improve determination efficiency of the caloric intake value of the user, in some embodiments, the food database may also be queried only N times. Specifically, when the “determining preprandial information of the food comprised in the picture based on the preprandial picture information of the food” is performed, the food database is queried using the method mentioned in the above specific examples. When the “determining postprandial information of the food comprised in the picture based on the postprandial picture information of the food” is performed, the food database is not queried.

Exemplarily, two method embodiments where postprandial information of the food comprised in the picture can be determined based on the postprandial picture information of the food without the aid of the food database are given below.

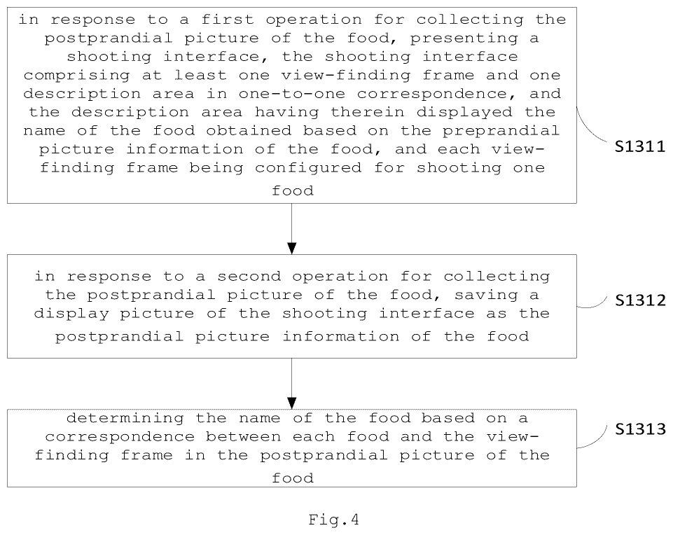

FIG. 4 is a flow diagram of a method for implementing S130 according to an embodiment of the present disclosure. Referring to FIG. 4, the method comprises: S1311, in response to a first operation for collecting the postprandial picture of the food, presenting a shooting interface. The shooting interface comprises at least one view-finding frame and one description area, the description area being in one-to-one correspondence with the view-finding frame, and the description area having therein displayed the name of the food obtained based on the preprandial picture information of the food; and each view-finding frame being used for shooting one food.

The first operation is used for triggering the terminal to enter a picture collection mode. In some embodiments, the terminal displays a SHOOT control, and when the user clicks on the SHOOT control, the terminal displays the shooting interface.

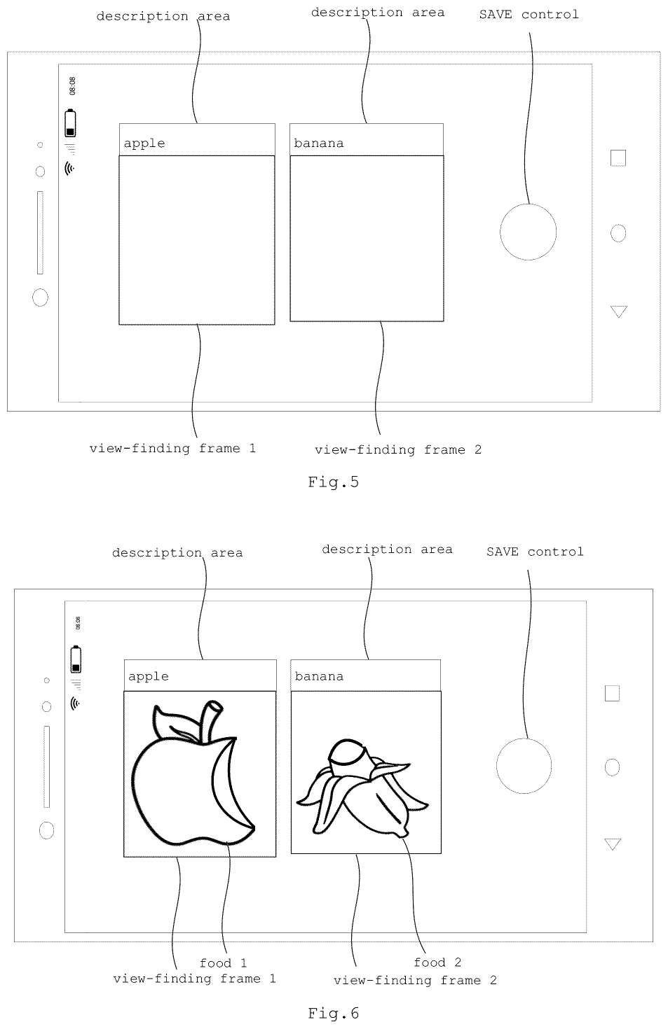

FIG. 5 is a schematic diagram of a shooting interface according to an embodiment of the present disclosure. Referring to FIG. 5, the shooting interface comprises two view-finding frames, each corresponding to one description area, in which a name of one food is presented. Referring to FIG. 2, the recognized preprandial picture of the food comprises apple and banana. In FIG. 5, the description area corresponding to a view-finding frame 1 has therein displayed “apple”, to prompt the user to shoot a postprandial apple using the view-finding frame 1. The description area corresponding to the view-finding frame 2 has therein displayed “banana” to prompt the user to shoot a postprandial banana using the view-finding frame 2.

The method further comprises: S1312, in response to a second operation for collecting the postprandial picture of the food, saving a display picture of the shooting interface as the postprandial picture information of the food.

The second operation is used for triggering the terminal to save the picture displayed by the display screen of the terminal, that is, picture collection is completed.

FIG. 6 is a schematic diagram of another shooting interface according to an embodiment of the present disclosure. Referring to FIG. 6, the user aims the view-finding frame 1 at the apple so that the view-finding frame 1 has therein displayed the apple. The user aims the view-finding frame 2 at the banana so that the view-finding frame 2 has therein displayed the banana. The user clicks on a SAVE control in the shooting interface so that the terminal saves the picture displayed by the terminal display screen.

The method further comprises: S1313, determining the name of the food based on a correspondence between each food and the view-finding frame in the postprandial picture of the food.

The essence of this step is to give the food name in the description area corresponding to the view-finding frame to the food collected by the view-finding frame. Exemplarily, continuing referring to FIG. 6, since the view-finding frame 1 is designated to shoot the apple, it is considered that food 1 collected by the view-finding frame 1 is the apple, and since the view-finding frame 2 is designated to shoot the banana, it is considered that food 2 collected by the view-finding frame 2 is the banana.

Since each frame is used for shooting a designated food and the designated food is a recognition result obtained after recognizing the preprandial picture of the food, that is, each frame is associated with data related to food designated for shooting, such as the name, calorific value per unit weight, and density of the food, the data related to the food having been obtained by querying the food database in the process of recognizing the preprandial picture of the food.

Subsequently, it is possible to obtain size information of the food in the postprandial picture of the food based on the postprandial picture of the food, and obtain volume information of the food based on the size information of the food; obtain the density and calorific value per unit weight of the food based on the data related to the food associated with the view-finding frame used for shooting the food; and obtain a weight of the food based on the volume information and density of the food, and obtain the caloric value of the food based on the weight and caloric value per unit weight of the food.

It should be emphasized that, if the view-finding frame 1 is designated to shoot the apple, but the user does not shoot the apple with the view-finding frame 1, but instead shoots the banana with the view-finding frame 1, the terminal would mistakenly mistake the banana for the apple, and mistakenly input the volume of the banana as an input into the algorithm. That is, in this solution, when the name of the food in the postprandial picture of the food is recognized, the accuracy of the recognition of the name depends on whether the user shoots according to the name of the food displayed in the description area corresponding to the view-finding frame 1.

According to the above technical solution, since it is not needed to query the food database again when the postprandial picture information of the food is recognized, it is possible to effectively improve the time spent on executing the method for determining caloric intake of a user, increase the response speed of the system, and enhance the user experience.

Based on the above technical solution, in some embodiments, after the S1311 and before the S1312, in response to a dragging operation on the view-finding frame, a position of the view-finding frame is changed so that the food displayed in the view-finding frame coincides with the name of the food displayed in the description area corresponding to the view-finding frame. Exemplarily, when the user shoots the apple by using the view-finding frame 1, he may initially aim the terminal camera at the apple, so that the apple appears in the terminal display screen, and then drag the view-finding frame 1, so that the apple is displayed in the view-finding frame 1. This setting is particularly applicable to a case where postprandial pictures are shot for at least two foods at the same time, which is because, in practice, at least two foods are placed at will, so that there is a very low chance that the two foods appear exactly in the view-finding frames to which they shall correspond respectively.

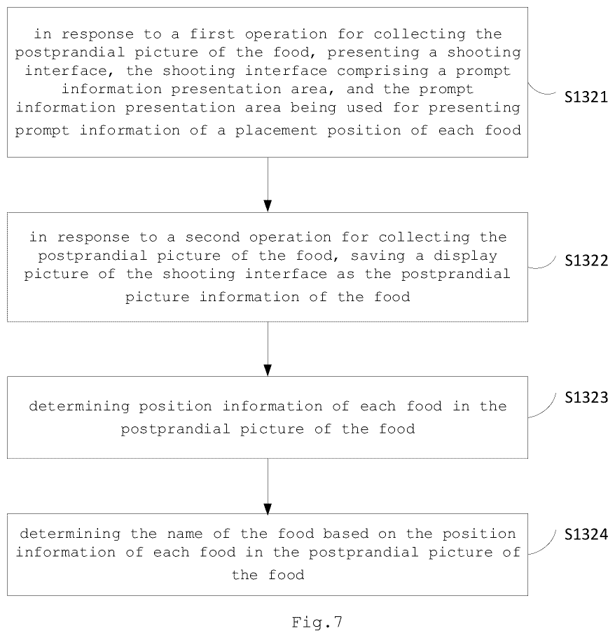

FIG. 7 is a flow diagram of another method for implementing S130 according to an embodiment of the present disclosure. Referring to FIG. 7, the method comprises: S1321, in response to a first operation for collecting the postprandial picture of the food, presenting a shooting interface, the shooting interface comprising a prompt information presentation area, and the prompt information presentation area being used for presenting prompt information of a placement position of each food.

The first operation is used for triggering the terminal to enter a picture collection mode, namely a shooting mode. In some embodiments, the terminal displays a SHOOT control, and when the user clicks on the SHOOT control, the terminal displays the shooting interface.

Content presented in the information presentation area is used for prompting the user how to place the food when shooting the postprandial food.

Information presented in the information presentation area can be text information or picture information.

If the information presented in the information presentation area is text information, exemplarily, if the recognized preprandial picture of the food comprises apple and banana, the prompt information presentation area has therein displayed “please place apple to the left of banana”.

If the information presented in the information presentation area is picture information, in some embodiments, the information presentation area has therein presented a thumbnail of the preprandial picture of the food.

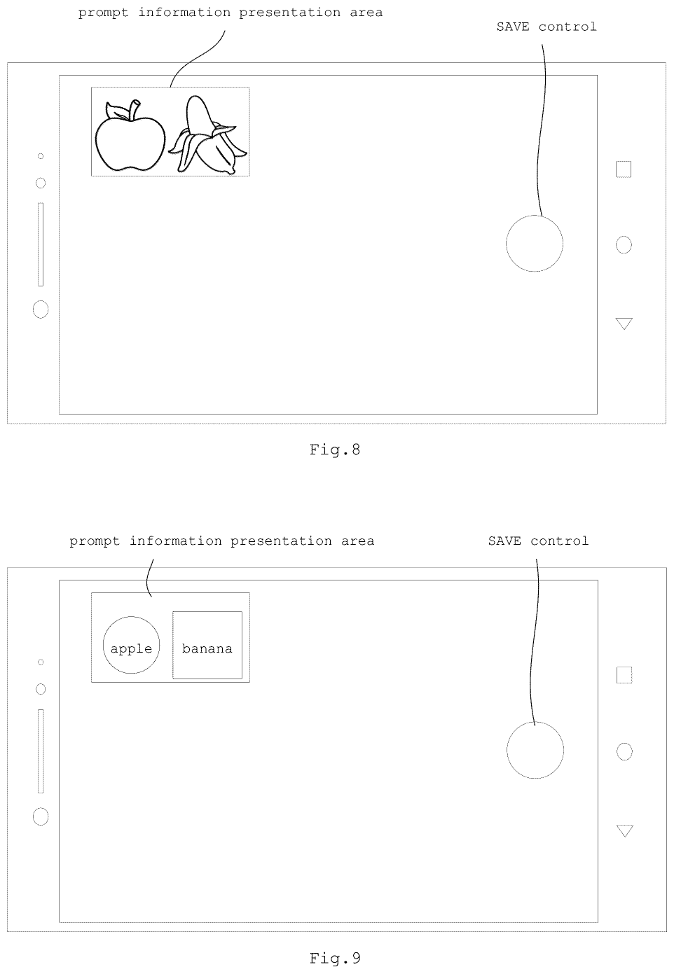

FIG. 8 is a schematic diagram of another shooting interface according to an embodiment of the present disclosure. Exemplarily, referring to FIG. 8, the prompt information presentation area is located in an upper left corner of the shooting interface. The prompt information presentation area has therein presented a thumbnail of the preprandial picture of the food.

Or, the prompt information presentation area has therein presented identification images of the foods, and a position relation between the identification images of the foods is consistent with a position relation between the foods in the preprandial picture of the food.

FIG. 9 is a schematic diagram of another shooting interface according to an embodiment of the present disclosure. Exemplarily, referring to FIG. 9, the prompt information presentation area is located in an upper left corner of the shooting interface. Since the recognized preprandial picture of the food comprises apple and banana, and the apple is located on the left and the banana is located on the right, a circle is used as an identification image of the apple, a rectangle is used as an identification image of the banana, and the circle is located on the left of the rectangle in the prompt information presentation area, so as to prompt that the user needs to place the residual apple to the left of the residual banana when shooting the postprandial picture.

It should be noted that, in the above examples, the prompt information presentation areas both are provided in the upper left corner of the shooting interface, which is merely a specific example of the present application and is not a limitation on the present application. In practice, the prompt information presentation area can be provided at any position of the shooting interface.

The method further comprises: S1322, in response to a second operation for collecting the postprandial picture of the food, saving a display picture of the shooting interface as the postprandial picture information of the food.

The second operation is used for triggering the terminal to save the picture displayed by the display screen of the terminal, that is, picture collection is completed.

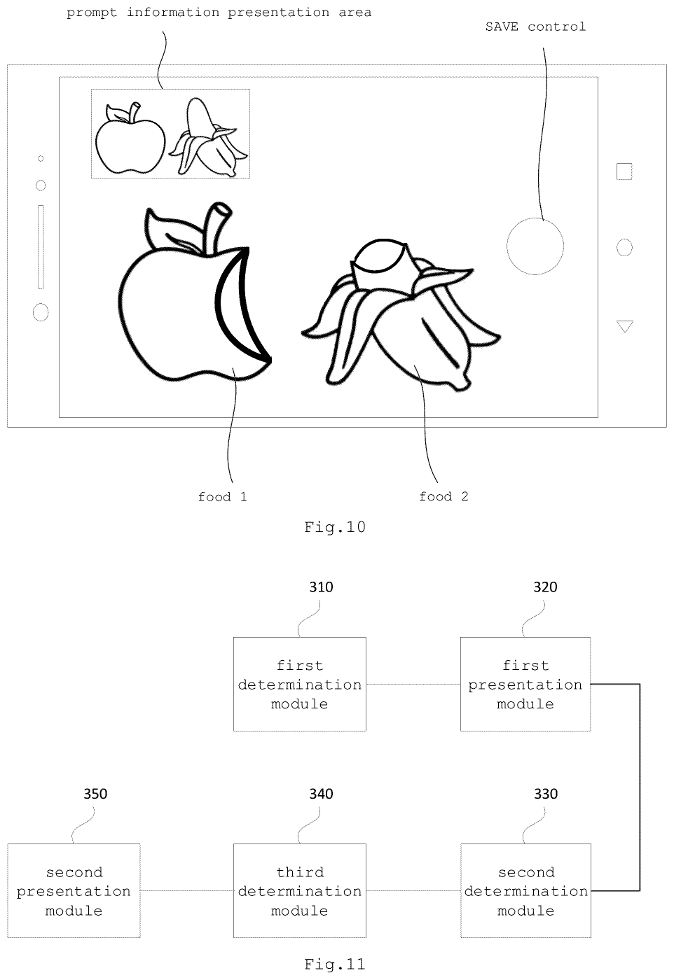

FIG. 10 is a schematic diagram of another shooting interface according to an embodiment of the present disclosure. Referring to FIG. 10, after placing the postprandial food according to the prompt information in the prompt information presentation area, the user aims the terminal camera at the postprandial food, so that the postprandial food appears in the display screen of the terminal. The user clicks on a SAVE control in the shooting interface, so that the terminal saves the picture displayed by the terminal display screen.

The method further comprises: S1323, determining position information of each food in the postprandial picture of the food; and S1324, determining the name of the food based on the position information of each food in the postprandial picture of the food.

Exemplarily, i.e., referring to FIG. 10, since it is unknown, before image recognition, whether the food 1 is an apple or banana and the food 2 is an apple or banana in the postprandial picture of the food, coordinate information of the food 1 as well as coordinate information of the food 2 in the postprandial picture of the food are acquired. A relative position relation between the food 1 and the food 2 can be obtained based on the coordinate data of the food 1 and food 2, for example, the food 1 is located on the left of the food 2, and since the user is instructed to place the apple on the left of the banana before shooting the postprandial food, it is considered that the food 1 is the apple and the food 2 is the banana.

And since the caloric value per unit weight and density of the apple in the food database have been obtained when S110 is executed, it is possible to subsequently obtain postprandial size information of the apple based on the postprandial picture of the apple, and obtain postprandial volume information of the apple based on the postprandial size information of the apple; and obtain a postprandial weight of the apple based on the postprandial volume information and density of the apple, and obtain a postprandial caloric value of the apple based on the postprandial weight and caloric value per unit weight of the apple. The way of obtaining a postprandial caloric value of the banana is similar to this, and will not be repeated herein.

It should be emphasized that, if the user places the food according to the information presented in the information presentation area, this can make the recognition accuracy of the postprandial picture of the food improved. That is, in this solution, when the name of the food in the postprandial picture of the food is recognized, the accuracy of the recognition of the name depends on whether the user places the food according to the information presented in the information presentation area when shooting.

According to the above technical solution, since it is not needed to query the food database again when the postprandial picture information of the food is recognized, it is possible to effectively improve the time spent on executing the method for determining caloric intake of a user, increase the response speed of the system, and enhance the user experience.

It should be noted that for the above method embodiments, for the sake of simple description, they are all described as combinations of a series of acts, but those skilled in the art should know that the present disclosure is not limited by the described order of acts, as certain steps may be performed in another order or simultaneously according to the present disclosure. Secondly, those skilled in the art should also know that the embodiments described in the description are all preferred embodiments and that acts and modules involved are not necessarily required for the present disclosure.

FIG. 11 is a schematic diagram of an apparatus for determining caloric intake of a user in an embodiment of the present disclosure. The apparatus for determining caloric intake of a user provided in the embodiment of the present disclosure can be configured in a client. Referring to FIG. 11, the apparatus for determining caloric intake of a user specifically comprises:
a first determination module 310 configured to acquire preprandial picture information of food, and determine a preprandial caloric value of the food based on the preprandial picture information of the food;
a first presentation module 320 configured to present a preprandial picture of the food, wherein the preprandial picture of the food is annotated with the preprandial caloric value of the food;
a second determination module 330 configured to acquire postprandial picture information of the food, and determine a postprandial caloric value of the food based on the postprandial picture information of the food;
a third determination module 340 configured to determine a caloric intake value of a user based on the preprandial caloric value and the postprandial caloric value of the food; and
a second presentation module 350 configured to present a postprandial picture of the food, wherein the postprandial picture of the food is annotated with one or more of the preprandial caloric value of the food, the postprandial caloric value of the food or the caloric intake value of the user.

Further, the first determination module 310 is configured to: determine preprandial information of the food comprised in the picture based on the preprandial picture information of the food, the preprandial information of the food comprising a name of the food and a preprandial weight of the food; and determine the preprandial caloric value of the food based on the preprandial information of the food.

The second determination module 330 is configured to: determine postprandial information of the food comprised in the picture based on the postprandial picture information of the food, the postprandial information of the food comprising a name of the food and a postprandial weight of the food; and determine the postprandial caloric value of the food based on the postprandial information of the food.

Further, the preprandial picture of the food comprises at least two foods. The second determination module 330 is configured to:
in response to a first operation for collecting the postprandial picture of the food, present a shooting interface, the shooting interface comprising at least one view-finding frame and one description area, the description area being in one-to-one correspondence with the view-finding frame, and the description area having therein displayed the name of the food obtained based on the preprandial picture information of the food; and each view-finding frame being used for shooting one food; and
in response to a second operation for collecting the postprandial picture of the food, save a display picture of the shooting interface as the postprandial picture information of the food.

Further, the second determination module 330 is configured to: after the in response to a first operation for collecting the postprandial picture of the food, presenting a shooting interface, and before the in response to a second operation for collecting the postprandial picture of the food, saving a display picture of the shooting interface as the postprandial picture information of the food, in response to a dragging operation on the view-finding frame, change a position of the view-finding frame, so that the food displayed in the view-finding frame is consistent with the name of the food displayed in the description area corresponding to the view-finding frame.

Further, the second determination module 330 is configured to: when the postprandial information is the name of the food, the determining the postprandial information of the food comprised in the picture based on the postprandial picture information of the food comprising:
determining the name of the food based on a correspondence between each food and the view-finding frame in the postprandial picture of the food.

Further, the preprandial picture of the food comprises at least two foods. The second determination module 330 is configured to:
in response to a first operation for collecting the postprandial picture of the food, present a shooting interface, the shooting interface comprising a prompt information presentation area, and the prompt information presentation area being used for presenting prompt information of placement positions of the foods; and
in response to a second operation for collecting the postprandial picture of the food, save a display picture of the shooting interface as the postprandial picture information of the food.

Further, the prompt information presentation area has therein presented a thumbnail of the preprandial picture of the food.

Further, the prompt information presentation area has therein presented identification images of the foods, and a position relation between the identification images of the foods is consistent with a position relation between the foods in the preprandial picture of the food.

Further, the second determination module 330 is configured to:
determine position information of each food in the postprandial picture of the food; and
based on the position information of each food in the postprandial picture of the food, determine the name of the food.

Further, the first presentation module 320 is configured to: acquire purchase information of the food; and
determine the information of the food based on the purchase information of the food and the preprandial picture information of the food, the information of the food comprising the name of the food, a brand of the food, and a weight of the food.

The apparatus for determining caloric intake of a user provided in the embodiment of the present disclosure can execute the steps executed by the client in the method for determining caloric intake of a user provided in the method embodiment of the present disclosure, and has beneficial effects of executing the steps, which are not repeated herein.

The above modules may be implemented as software components executed on one or more general-purpose processors, or as hardware executing certain functions or combinations thereof, such as a programmable logic device and/or application specific integrated circuit. In some embodiments, these modules may be embodied in a form of a software product, which may be stored in non-volatile storage media comprising instructions that cause a computer device (e.g., a personal computer, server, network device, mobile terminal, etc.) to implement the method described in the embodiment of the present disclosure. In other embodiments, the above modules may also be implemented on a single device or may be distributed on a plurality of devices. Functions of these modules may be combined with each other, or further divided into a plurality of units.

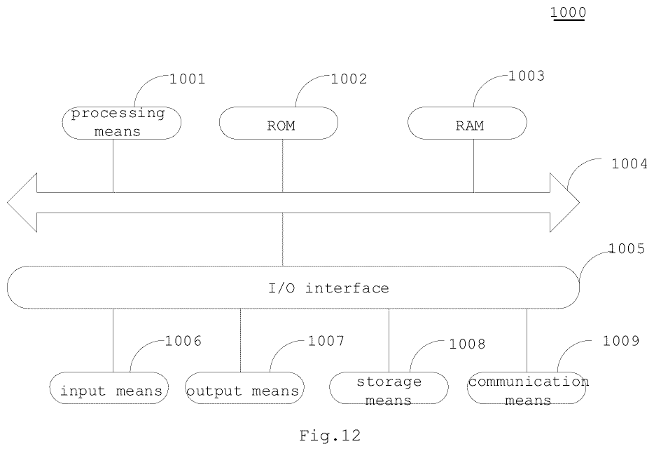

FIG. 12 is a schematic structural diagram of an electronic device in an embodiment of the present disclosure. Hereinafter, reference is made specifically to FIG. 12, which illustrates a schematic structural diagram of an electronic device 1000 suitable for implementing the embodiment of the present disclosure. The electronic device 1000 in the embodiment of the present disclosure may comprise, but is not limited to, a mobile terminal such as a mobile phone, laptop computer, digital broadcast receiver, PDA (Personal Digital Assistant), PAD (Portable Android Device), PMP (Portable Multimedia Player), vehicle-mounted terminal (e.g., vehicle-mounted navigation terminal), and wearable electronic device, and a fixed terminal such as a digital TV, desktop computer, and smart home appliance. The electronic device shown in FIG. 12 is only an example, and should not bring any limitation to the functions and the scope of use of the embodiments of the present disclosure.

As shown in FIG. 12, the electronic device 1000 may comprise a processing means (e.g., a central processing unit, a graphics processing unit, etc.) 1001 that may perform various appropriate actions and processes according to a program stored in a read-only memory (ROM) 1002 or a program loaded from a storage means 1008 into a random access memory (RAM) 1003 to implement the method for determining caloric intake of a user according to the embodiment of the present disclosure. In the RAM 1003, various programs and information required for the operation of the electronic device 1000 are also stored. The processing means 1001, ROM 1002, and RAM 1003 are connected to each other by a bus 1004. An input/output (I/O) interface 1005 is also connected to the bus 1004.

Generally, the following means may be connected to the I/O interface 1005: an input means 1006 comprising, for example, a touch screen, touch pad, keyboard, mouse, camera, microphone, accelerometer, gyroscope, and the like; an output means 1007 comprising, for example, a liquid crystal display (LCD), speaker, vibrator, and the like; the storage means 1008 comprising, for example, a magnetic tape, hard disk, and the like; and a communication means 1009. The communication means 1009 may allow the electronic device 1000 to communicate wirelessly or by wire with other devices to exchange information. While FIG. 12 illustrates the electronic device 1000 having various means, it should be understood that not all illustrated means are required to be implemented or provided. More or fewer means may be alternatively implemented or provided.

In particular, according to the embodiments of the present disclosure, the above processes described with reference to the flow diagrams may be implemented as a computer software program. For example, an embodiment of the present disclosure comprises a computer program product, which comprises a computer program carried on a non-transitory computer-readable medium, the computer program containing program code for performing the method illustrated by the flow diagrams, thereby implementing the method for determining caloric intake of a user as described above. In such an embodiment, the computer program may be downloaded and installed from the network through the communication means 1009, or installed from the storage means 1008, or installed from the ROM 1002. The computer program, when executed by the processing means 1001, performs the above functions defined in the method of the embodiment of the present disclosure.

It should be noted that the above computer-readable medium of the present disclosure may be a computer-readable signal medium or a computer-readable storage medium or any combination of the above two. The computer-readable storage medium may be, for example, but is not limited to, an electronic, magnetic, optical, electromagnetic, infrared, or semiconductor system, apparatus, or device, or any combination of the foregoing. More specific examples of the computer-readable storage medium may comprise, but are not limited to: an electrical connection having one or more wires, a portable computer diskette, a hard disk, a random access memory (RAM), a read-only memory (ROM), an erasable programmable read-only memory (EPROM or flash memory), an optical fiber, a portable compact disc read-only memory (CD-ROM), an optical storage device, a magnetic storage device, or any suitable combination of the foregoing. In the present disclosure, the computer-readable storage medium may be any tangible medium containing or storing a program, wherein the program can be used by or in conjunction with an instruction execution system, apparatus, or device. However, in the present disclosure, the computer-readable signal medium may comprise an information signal propagated in baseband or as part of a carrier wave, in which computer-readable program code is carried. Such a propagated information signal may take a variety of forms, comprising, but not limited to, an electromagnetic signal, optical signal, or any suitable combination of the forgoing. The computer-readable signal medium may also be any computer-readable medium other than the computer-readable storage medium, wherein the computer-readable signal medium can send, propagate, or transmit a program for use by or in conjunction with an instruction execution system, apparatus, or device. The program code contained on the computer-readable medium may be transmitted using any appropriate medium, comprising but not limited to: a wire, an optical cable, RF (Radio Frequency), etc., or any suitable combination of the foregoing.

In some implementations, a client and a server may communicate using any currently known or future developed network protocol, such as HTTP (HyperText Transfer Protocol), and may be interconnected with any form or medium of digital information communication (e.g., a communication network). Examples of the communication network comprise a local area network (“LAN”), a wide area network (“WAN”), an internet (e.g., the Internet), and a peer-to-peer network (e.g., an ad hoc peer-to-peer network), as well as any currently known or future developed network.

The above computer-readable medium may be contained in the above electronic device; or may exist separately without being assembled into the electronic device.

The above computer-readable medium has one or more programs carried thereon, which, when executed by the electronic device, cause the electronic device to:
acquire preprandial picture information of food, and determine a preprandial caloric value of the food based on the preprandial picture information of the food;
present a preprandial picture of the food, wherein the preprandial picture of the food is annotated with the preprandial caloric value of the food;
acquire postprandial picture information of the food, and determine a postprandial caloric value of the food based on the postprandial picture information of the food;
determine a caloric intake value of a user based on the preprandial caloric value and the postprandial caloric value of the food; and
present a postprandial picture of the food, wherein the postprandial picture of the food is annotated with one or more of the preprandial caloric value of the food, the postprandial caloric value of the food or the intake caloric value of the user.

In some embodiments, when the above one or more programs are executed by the electronic device, the electronic device may also perform other steps described in the above embodiments.

Computer program code for performing the operation of the present disclosure may be written in one or more programming languages or a combination thereof, wherein the above programming language comprises but is not limited to an object-oriented programming language such as Java, Smalltalk, and C++, and also comprises a conventional procedural programming language, such as a “C” language or a similar programming language. The program code may be executed entirely on a user's computer, partly on a user's computer, as a stand-alone software package, partly on a user's computer and partly on a remote computer, or entirely on a remote computer or server. In a scenario where a remote computer is involved, the remote computer may be connected to a user's computer through any type of network, comprising a local area network (LAN) or a wide area network (WAN), or may be connected to an external computer (for example, through the Internet using an Internet service provider).

The flow diagrams and block diagrams in the drawings illustrate the possibly implemented architecture, functions, and operations of the system, method and computer program product according to various embodiments of the present disclosure. In this regard, each block in the flow diagrams or block diagrams may represent a module, program segment, or portion of code, which comprises one or more executable instructions for implementing a specified logical function. It should also be noted that, in some alternative implementations, functions noted in blocks may occur in a different order from those noted in the drawings. For example, two blocks shown in succession may, in fact, be executed substantially concurrently, or they may sometimes be executed in a reverse order, which depends upon the functions involved. It will also be noted that each block in the block diagrams and/or flow diagrams, and a combination of the blocks in the block diagrams and/or flow diagrams, can be implemented by a special-purpose hardware-based system that performs specified functions or operations, or by a combination of special-purpose hardware and computer instructions.

The involved units described in the embodiments of the present disclosure may be implemented by software or hardware. The name of the unit does not, in some cases, constitute a limitation on the unit itself.

The functions described above herein may be executed, at least partially, by one or more hardware logic components. For example, without limitation, a hardware logic component of an exemplary type that may be used comprises: a field programmable gate array (FPGA), an application specific integrated circuit (ASIC), an application specific standard parts (ASSP), a system on chip (SOC), a complex programmable logic device (CPLD), and the like.

In the context of this disclosure, a machine-readable medium may be a tangible medium, which can contain, or store a program for use by or in conjunction with an instruction execution system, apparatus, or device. The machine-readable medium may be a machine-readable signal medium or a machine-readable storage medium. The machine-readable medium may comprise, but is not limited to, an electronic, magnetic, optical, electromagnetic, infrared, or semiconductor system, apparatus, or device, or any suitable combination of the foregoing. More specific examples of the machine-readable storage medium comprise an electrical connection based on one or more wires, a portable computer diskette, a hard disk, a random access memory (RAM), a read-only memory (ROM), an erasable programmable read-only memory (EPROM or flash memory), an optical fiber, a portable compact disc read-only memory (CD-ROM), an optical storage device, a magnetic storage device, or any suitable combination of the foregoing.

According to one or more embodiments of the present disclosure, there is provided an electronic device, comprising:
one or more processors; and
a memory configured to store one or more programs,
the one or more programs, when executed by the one or more processors, causing the one or more processors to implement any of the methods for determining caloric intake of a user according to the present disclosure.

According to one or more embodiments of the present disclosure, there is provided a computer-readable storage medium having thereon stored a computer program which, when executed by a processor, implements any of the methods for determining caloric intake of a user according to the present disclosure.

An embodiment of the present disclosure further provides a computer program product comprising a computer program or instructions which, when executed by a processor, implement the method for determining caloric intake of a user as described above.

It should be noted that, relational terms such as “first” and “second”, herein, are only used for distinguishing one entity or operation from another entity or operation without necessarily requiring or implying any such actual relation or order between these entities or operations. Moreover, the term “comprise”, “comprise”, or any other variation thereof, is intended to encompass a non-exclusive inclusion, such that a process, method, article, or device comprising a list of elements not only comprises those elements but also comprises other elements not expressly listed, or also comprises elements inherent to such a process, method, article, or device. Without more limitations, an element defined by a statement “comprising a . . . ” does not exclude the presence of another identical element in a process, method, article, or device that comprises the element.

The above only describes specific implementations of the present disclosure, to enable those skilled in the art to understand or implement the present disclosure. Various modifications to these embodiments will be apparent to those skilled in the art, and the general principles defined herein may be implemented in other embodiments without departing from the spirit or scope of the present disclosure. Therefore, the present disclosure will not be limited to these embodiments described herein, but conform to the widest scope consistent with the principles and novel features disclosed herein.

1. A method of determining caloric intake of a user, comprising:
acquiring preprandial picture information of food, and determining a preprandial caloric value of the food based on the preprandial picture information of the food;
presenting a preprandial picture of the food, wherein the preprandial picture of the food is annotated with the preprandial caloric value of the food;
acquiring postprandial picture information of the food, and determining a postprandial caloric value of the food based on the postprandial picture information of the food;
determining a caloric intake value of a user based on the preprandial caloric value and the postprandial caloric value of the food; and
presenting a postprandial picture of the food, wherein the postprandial picture of the food is annotated with one or more of the preprandial caloric value of the food, the postprandial caloric value of the food, or the caloric intake value of the user.

2. The method according to claim 1, wherein:
the determining a preprandial caloric value of the food based on the preprandial picture information of the food comprises: determining preprandial information of the food comprised in the picture based on the preprandial picture information of the food, the preprandial information of the food comprising a name of the food and a preprandial weight of the food; and determining the preprandial caloric value of the food based on the preprandial information of the food; and
the determining a postprandial caloric value of the food based on the postprandial picture information of the food comprises: determining postprandial information of the food comprised in the picture based on the postprandial picture information of the food, the postprandial information of the food comprising a name of the food and a postprandial weight of the food; and determining the postprandial caloric value of the food based on the postprandial information of the food.

3. The method according to claim 2, wherein the preprandial picture of the food comprises at least two foods, the acquiring postprandial picture information of the food comprising:
in response to a first operation for collecting the postprandial picture of the food, presenting a shooting interface, the shooting interface comprising at least one view-finding frame and one description area in one-to-one correspondence, and the description area having therein displayed the name of the food obtained based on the preprandial picture information of the food, and each view-finding frame being configured for shooting one food; and
in response to a second operation for collecting the postprandial picture of the food, saving a display picture of the shooting interface as the postprandial picture information of the food.

4. The method according to claim 3, comprising:
after the presenting the shooting interface, and before the saving the display picture of the shooting interface as the postprandial picture information of the food, in response to a dragging operation on the view-finding frame, changing a position of the view-finding frame, so that the food displayed in the view-finding frame is consistent with the name of the food displayed in the description area corresponding to the view-finding frame.

5. The method according to claim 3, wherein the postprandial information is the name of the food, the determining postprandial information of the food comprised in the picture based on the postprandial picture information of the food comprising:
determining the name of the food based on the correspondence between each food and the view-finding frame in the postprandial picture of the food.

6. The method according to claim 2, wherein the preprandial picture of the food comprises at least two foods, the acquiring postprandial picture information of the food comprising:
in response to a first operation for collecting the postprandial picture of the food, presenting a shooting interface, the shooting interface comprising a prompt information presentation area for presenting prompt information of placement positions of the foods; and
in response to a second operation for collecting the postprandial picture of the food, saving a display picture of the shooting interface as the postprandial picture information of the food.

7. The method according to claim 6, wherein the prompt information presentation area has therein presented a thumbnail of the preprandial picture of the food.

8. The method according to claim 6, wherein the prompt information presentation area has therein presented identification images of the foods, and a position relation between the identification images of the foods is consistent with a position relation between the foods in the preprandial picture of the food.

9. The method according to claim 6, wherein the postprandial information is the name of the food, the determining postprandial information of the food comprised in the picture based on the postprandial picture information of the food comprising:
determining position information of each food in the postprandial picture of the food;
and
determining the name of the food based on the position information of each food in the postprandial picture of the food.

10. The method according to claim 2, wherein the determining information of the food comprised in the picture based on the preprandial picture information of the food, the information of the food comprising a name of the food and a weight of the food, comprises:
acquiring purchase information of the food; and
determining the information of the food based on the purchase information of the food and the preprandial picture information of the food, the information of the food comprising the name of the food, a brand of the food, and a weight of the food.

11. The method according to claim 2, wherein the determining information of the food comprised in the picture based on the preprandial picture information of the food comprises:
pre-constructing a food database, the food database comprising a plurality of pieces of information, each comprising a name of food, a calorific value per unit weight and density of the food, and a picture of the food;
comparing the preprandial picture of the food with the information in the food database to obtain data hit with the food in the preprandial picture of the food;
determining the name, caloric value per unit weight, and density of the food comprised in the preprandial picture of the food based on the hit data;
obtaining size information of the food in the preprandial picture of the food based on the preprandial picture of the food, and obtaining volume information of the food based on the size information of the food; and
obtaining a weight of the food based on the volume information and density of the food, and obtaining the caloric value of the food based on the weight and caloric value per unit weight of the food.

12. The method according to claim 2, wherein the determining postprandial information of the food comprised in the picture based on the postprandial picture information of the food comprises:
pre-constructing a food database, the food database comprising a plurality of pieces of information, each comprising a name of food, a calorific value per unit weight and density of the food, and a picture of the food;
comparing the postprandial picture of the food with the information in the food database to obtain data hit with the food in the postprandial picture of the food;
determining the name, caloric value per unit weight, and density of the food comprised in the postprandial picture of the food based on the hit data;
obtaining size information of the food in the postprandial picture of the food based on the postprandial picture of the food, and obtaining volume information of the food based on the size information of the food; and
obtaining a weight of the food based on the volume information and density of the food, and obtaining the caloric value of the food based on the weight and caloric value per unit weight of the food.

13. The method according to claim 1, further comprising:
correcting the caloric intake value of the user with an error parameter, and taking the corrected caloric intake value of the user as a final caloric intake value of the user.

14. The method according to claim 6, wherein the prompt information presented in the prompt information presentation area comprises text information.

15. (canceled)

16. An electronic device, comprising:
one or more processors; and
a storage means configured to store one or more programs, the one or more programs, when executed by the one or more processors, causing the one or more processors to a method of determining caloric intake of a user, comprising:
acquiring preprandial picture information of food, and determining a preprandial caloric value of the food based on the preprandial picture information of the food;
presenting a preprandial picture of the food, wherein the preprandial picture of the food is annotated with the preprandial caloric value of the food;
acquiring postprandial picture information of the food, and determining a postprandial caloric value of the food based on the postprandial picture information of the food;
determining a caloric intake value of a user based on the preprandial caloric value and the postprandial caloric value of the food; and
presenting a postprandial picture of the food, wherein the postprandial picture of the food is annotated with one or more of the preprandial caloric value of the food, the postprandial caloric value of the food, or the caloric intake value of the user.

17. A non-transitory computer-readable storage medium having thereon stored a computer program which, when executed by a processor, implements a method of determining caloric intake of a user, comprising:
acquiring preprandial picture information of food, and determining a preprandial caloric value of the food based on the preprandial picture information of the food;
presenting a preprandial picture of the food, wherein the preprandial picture of the food is annotated with the preprandial caloric value of the food;
acquiring postprandial picture information of the food, and determining a postprandial caloric value of the food based on the postprandial picture information of the food;
determining a caloric intake value of a user based on the preprandial caloric value and the postprandial caloric value of the food; and
presenting a postprandial picture of the food, wherein the postprandial picture of the food is annotated with one or more of the preprandial caloric value of the food, the postprandial caloric value of the food, or the caloric intake value of the user.

18. (canceled)

19. The electronic device according to claim 16, wherein:
the determining a preprandial caloric value of the food based on the preprandial picture information of the food comprises: determining preprandial information of the food comprised in the picture based on the preprandial picture information of the food, the preprandial information of the food comprising a name of the food and a preprandial weight of the food; and determining the preprandial caloric value of the food based on the preprandial information of the food; and
the determining a postprandial caloric value of the food based on the postprandial picture information of the food comprises: determining postprandial information of the food comprised in the picture based on the postprandial picture information of the food, the postprandial information of the food comprising a name of the food and a postprandial weight of the food; and determining the postprandial caloric value of the food based on the postprandial information of the food.

20. The electronic device according to claim 19, wherein the preprandial picture of the food comprises at least two foods, the acquiring postprandial picture information of the food comprising:
in response to a first operation for collecting the postprandial picture of the food, presenting a shooting interface, the shooting interface comprising at least one view-finding frame and one description area in one-to-one correspondence, and the description area having therein displayed the name of the food obtained based on the preprandial picture information of the food, and each view-finding frame being configured for shooting one food; and
in response to a second operation for collecting the postprandial picture of the food, saving a display picture of the shooting interface as the postprandial picture information of the food.

21. The non-transitory computer-readable storage medium according to claim 17, wherein:
the determining a preprandial caloric value of the food based on the preprandial picture information of the food comprises: determining preprandial information of the food comprised in the picture based on the preprandial picture information of the food, the preprandial information of the food comprising a name of the food and a preprandial weight of the food; and determining the preprandial caloric value of the food based on the preprandial information of the food; and
the determining a postprandial caloric value of the food based on the postprandial picture information of the food comprises: determining postprandial information of the food comprised in the picture based on the postprandial picture information of the food, the postprandial information of the food comprising a name of the food and a postprandial weight of the food; and determining the postprandial caloric value of the food based on the postprandial information of the food.

22. The non-transitory computer-readable storage medium according to claim 21, wherein the preprandial picture of the food comprises at least two foods, the acquiring postprandial picture information of the food comprising:
in response to a first operation for collecting the postprandial picture of the food, presenting a shooting interface, the shooting interface comprising at least one view-finding frame and one description area in one-to-one correspondence, and the description area having therein displayed the name of the food obtained based on the preprandial picture information of the food, and each view-finding frame being configured for shooting one food; and
in response to a second operation for collecting the postprandial picture of the food, saving a display picture of the shooting interface as the postprandial picture information of the food.
